(19) United States
(12) Patent Application Publication
(10) Pub. No.: US 2026/0139299 A1
(43) Pub. Date: May 21, 2026

(54) METHODS OF FABRICATING HIGH-THROUGHPUT ARRAYS WITH CUSTOMIZABLE PROPERTIES USING SILICA MICROSPHERE ASSISTED PATTERNING

(71) Applicant: Arizona Board of Regents on Behalf of Arizona State University, Scottsdale, AZ (US)

(72) Inventors: Prathamesh CHOPADE, Tempe, AZ (US); Rizal HARIADI, Phoenix, AZ (US); Deeksha SATYABOLA, Tempe, AZ (US); Aaron SAKAI, Phoenix, AZ (US)

(21) Appl. No.: 19/396,193

(22) Filed: Nov. 20, 2025

Related U.S. Application Data
(60) Provisional application No. 63/722,979, filed on Nov. 20, 2024.

(51) Int. Cl. C12Q 1/6837

(52) U.S. Cl. CPC C12Q 1/6837

(57) ABSTRACT

The described method leverages nanosphere lithography (NSL) using nanoparticles having Young's modulus of at least 5 GPa, such as silica beads, to create high-density, periodic DNA origami nanoarrays on glass surfaces. In some aspects, by employing 1 μm silica beads, which have a higher Young's modulus compared to polystyrene beads, the sticky patch size (100 nm) is spaced out by 1 μm while maintaining DNA origami structures per spot. The resulting nanoarray consists of sticky patches with single DNA origami. This clean room-free fabrication method significantly reduces the cost per chip, while enabling precise positioning of target molecules at high throughput.

### RELATED APPLICATIONS

This application claims priority to and the benefit of U.S. provisional patent application 63/722,979, filed Nov. 20, 2024, titled “Methods of Fabricating High-Throughput Arrays with Customizable Properties Using Silica Microsphere Assisted Patterning,” the entirety of the disclosure of which is hereby incorporated by this reference.

### STATEMENT REGARDING FEDERALLY SPONSORED RESEARCH OR DEVELOPMENT

This invention was made with government support under 2227650 awarded by the National Science Foundation. The government has certain rights in the invention.

### TECHNICAL FIELD

This document relates to fabrication of high-throughput arrays.

### BACKGROUND

In terms of high-throughput biomolecular detection platforms, current technologies use processes like electron beam lithography (EBL), focused ion beam (FIB), and associated processes that must be performed in a cleanroom. These processes are complex, time consuming and often involve the use of corrosive materials that can compromise the health in the long run. Although the products made by using such processes have user-desired features with precise control over fabrication, sustainability is a major issue yet to be addressed. Furthermore, such needs require higher operation and maintenance costs which drives up the product cost making affordability a concern. Emerging technology, such as microfluidic platforms is an offer promising alternative. Microfluidics, for instance, allows high-throughput screening with reduced reagent volumes and enhanced control over experimental conditions. But fabricating microfluidic devices is complex and require imprinting from molds made by EBL or photolithography.

In the context of biomolecule detection platform, the trade-off between concentration and throughput poses a significant limitation for conventional single-molecule studies. Usually, a balance between avoiding molecular overlap and maintaining enough observable molecules needs to be preserved for better data quality. User-defined concentration with high-throughput can be promised using nanostencil lithograph (NSL) by positioning molecules on a surface in a close-packing for optimal data quality. Methods such as EBL and photolithography have been employed to create high-density nanoarrays for deterministic positioning of molecules at the diffraction limit. These methods are lengthy, limits their use due to complex steps and harsh chemicals involved. Although fabricating arrays with NSL overcomes these disadvantages in a relatively simple way, array quality is compromised if the colloidal mask is inconsistent. Furthermore, periodicity of NSL-made arrays strongly depends on the particle size, and it becomes crucial to space out the molecules for fluorescence-based readouts. This can be achieved by close packing the molecules at the diffraction limit of light, but at higher periodicity maintaining patches compatible to the molecule dimensions to ensure single occupancy is challenging in NSL.

In view of the foregoing, improve methods of masking an array surface in preparation for lithography are needed.

### SUMMARY

In one aspect, the techniques described herein relate to a method of masking an array surface in preparation for lithography comprising: combining silica nanoparticles with an organic solvent; sonicating the combination of silica nanoparticles and the organic solvent to produce colloidal silica crystals; dropping the colloidal silica crystals on a glass surface that is submerged in water; and drying the colloidal silica crystals droplets on the glass surface thereby producing a silica film over the glass surface.

In some implementations, the method further comprises covering the silica film with a silane. In some aspects, the silane is an organosilane. In some implementations, the silane is a perfluorinated compound. In particular implementations, the silane is HMDS or TMFDS.

In another aspect, the techniques described herein relate to a method of preparing a glass surface for printing an array comprising: combining silica nanoparticles with an organic solvent; sonicating the combination of silica nanoparticles and the organic solvent to produce colloidal silica crystals; dropping the colloidal silica crystals on a glass surface that is submerged in water; drying the colloidal silica crystals droplets on the glass surface thereby producing a silica film over the glass surface; and covering the glass surface having the silica film with a silane to produce a silane-modified glass surface. In some aspects, the silane is an organosilane such as HMDS or TMFDS. In other aspects, the silane is a perfluorinated compound.

In some implementations, the method further comprises removing the silica film from the glass surface, for example by sonicating silane-modified glass surface. In some implementations, the method further comprises heating the silane-modified glass surface after removing the silica film. In certain embodiments, the methods described herein further comprises confirming presence of layer of silica nanospheres on the glass surface via diffraction analysis.

In some embodiments, the silica nanoparticles have diameters of 1 um. Thus, in such embodiments, the silane-modified glass surface includes sticky patches spaced out by 1 um. In some aspects, each sticky patch has a diameter of 100 nm.

In yet another aspect, the techniques described herein relate to a method of masking an array surface in preparation for lithography including combining nanoparticles having Young's modulus of at least 5 GPa with an organic solvent; sonicating the combination of the nanoparticles and the organic solvent to produce colloidal crystals; dropping the colloidal crystals on a glass surface that is submerged in water; and drying the colloidal crystals droplets on the glass surface thereby producing a nanoparticle film over the glass surface. In some implementations, the method further comprises covering the nanoparticle film with a silane (such as HMDS or TMFDS).

In still another aspects, the techniques described herein relate to a method of preparing a glass surface for printing an array comprising: combining nanoparticles having Young's modulus of at least 5 GPa with an organic solvent; sonicating the combination of nanoparticles and the organic solvent to produce colloidal crystals; dropping the colloidal crystals on a glass surface that is submerged in water; drying the colloidal crystals droplets on the glass surface thereby producing a nanoparticle film over the glass surface; and covering the glass surface having the nanoparticle film with a silane to produce a silane-modified glass surface.

In some implementations, the method further comprises removing the nanoparticle film from the glass surface, for example by sonicating silane-modified glass surface. In some implementations, the method further comprises heating the silane-modified glass surface after removing the nanoparticle film. In some aspects, the method further comprises confirming presence of a layer of nanospheres on the glass surface via diffraction analysis thereby indicating the presence of the nanoparticle film over the glass surface.

In some embodiments of preparing a glass surface for printing an array, the nanoparticles are nanoparticles of an oxide having Young's modulus of about 50-200 GPa, for example, aluminum oxide, zirconium oxide, or titanium oxide. In certain embodiments, the nanoparticles have Young's modulus of about 10 GPa, for example, the nanoparticles are silica nanoparticles.

In some aspects, the techniques described herein relate to a method of printing a customizable DNA array comprising: combining nanoparticles having Young's modulus of at least 5 GPa with an organic solvent; sonicating the combination of nanoparticles and the organic solvent to produce colloidal crystals; dropping the colloidal crystals on a glass surface that is submerged in water; drying the colloidal crystals droplets on the glass surface thereby producing a nanoparticle film over the glass surface; covering the glass surface having the nanoparticle film with a silane to produce a modified glass surface; removing the nanoparticle film from the modified glass surface to produce a glass surface including binding sites; and incubating DNA origami with the glass surface including binding sites thereby hydrolyzing the silane and the DNA origami. In some aspects, the silane is an organosilane such as HMDS or TMFDS. In other aspects, the silane is a perfluorinated compound.

In some aspects, the method further comprises confirming presence of a layer of nanospheres on the glass surface via diffraction analysis thereby indicating the presence of the nanoparticle film over the glass surface.

In some embodiments of the method of printing a customizable DNA array, the nanoparticles are nanoparticles of an oxide having Young's modulus of about 50-200 GPa, for example, aluminum oxide, zirconium oxide, or titanium oxide. In certain implementations, the nanoparticles have Young's modulus of about 10 GPa. For example, the nanoparticles are silica nanoparticles. In some aspects, the silica nanoparticles have diameters of 1 um. In such implementations, the customizable DNA array is spaced out by 1 um.

In some implementations of the techniques described herein, the organic solvent is N,N-dimethylformamide anhydrous (DMF) and/or dichloromethane (DCM).

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

FIG. 1A is a schematic of the process to create a binding site.

FIG. 1B is a schematic of the process for DNA origami placement and washing.

FIG. 2 compares the structure of silica and polystyrene nanoparticles.

FIG. 3 is an exemplary DNA-PAINT image of the DNA nanoarray produced using SIMPLE.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, the term “DNA origami” refers to either two-dimensional (2D) or three-dimensional (3D) shapes formed by folded DNA. In other words, a DNA origami is formed by nanoscale folding to DNA. In some aspects, the dimensions of the DNA origami is 50-100 nm. In certain implementations, an array comprising 3D DNA origami is produced by assembling the 3D structure from using a 2D DNA origami as a pedestal. For example, the 2D DNA origami is attached to the sticky patch and then the 3D DNA origami is assembled from the attached 2D DNA origami.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Disclosed herein are methods related to making customized arrays, including methods of preparing a surface for printing an array. In some embodiments, methods of masking an array surface in preparation for lithography, methods of preparing a glass surface for printing an array, and method of printing a customizable DNA array are described. In particular, the methods have unique capabilities in terms of precise positioning, improved loading efficiency, and cost-effective, high-throughput production of DNA origami nanoarrays.

Colloidal self-assembly provides a facile method to fabricate opals or crystals with nanoscale features that can be used to make arrays with nanosphere lithography (NSL). The method does not require the use of cleanrooms. The quality of nanoarrays is heavily influenced by the features of the colloidal crystals formed via self-assembly, which relies on the particle's ability to remain suspended in solution. Moreover, the quality of monolayers of nanoparticles formed by techniques such as spin coating (requires fine tuning of several parameters) and drop casting (suffers from coffee ring effect) replies on particle solubility. Aggregates, when placed on surface, severely affect monolayer formation, which subsequently affects the feature and array quality. Additionally, current opal fabrication techniques only produce high-quality structures over a limited range of sphere sizes or require complex processes and equipment. Described herein is a method that deposits colloidal silica crystals self-assembled at the air-water interface by a reverse Langmuir-Schaefer method, which is able to overcome above mentioned limitations. This method is referred to herein as “SIMPLE”, Silica Microspheres assisted Patterning via Liquid Ejection. However, nanoparticles of other stiff material (for example, materials with Young's modulus of at least 5 GPa) may also be used as colloids. Other than silica, nanoparticles of aluminum oxide, zirconium oxide, and titanium oxide are suitable stiff materials for the methods described herein. In some aspects, nanoparticles of metal oxides with Young's modulus of 50-200 GPa are the nanoparticles of stiff materials used in the methods described herein.

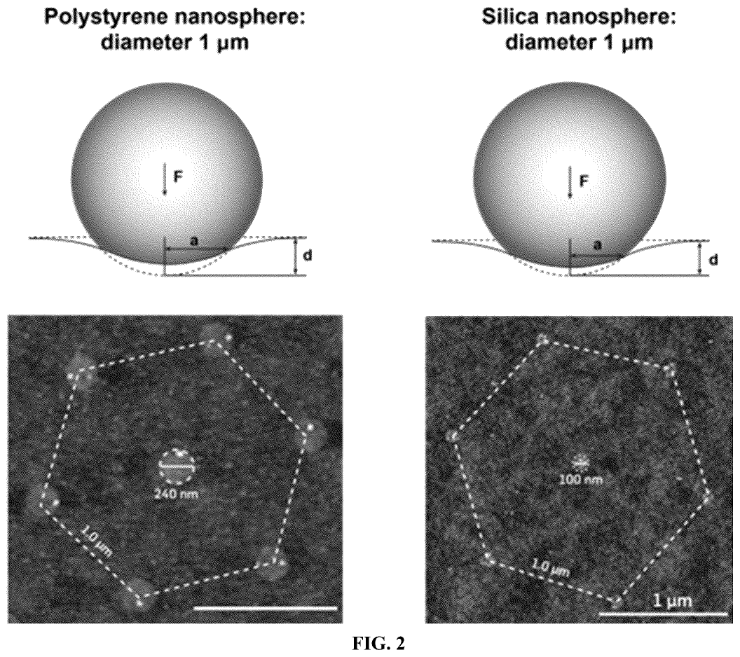

SIMPLE comprises fabricating high quality colloidal crystals to be used as masks when fabricating single molecule array on an enriched glass substrate. SIMPLE uses nanomaterials with different Young's modulus to ensure compatible patches with variable periodicity and improves sensitivity, specificity in single molecule measurements. Silica nanoparticles have a higher Young's modulus (˜10 GPa) compared to polystyrene nanoparticles (˜0.5 GPa) that are routinely used to fabricate nanoarrays (FIG. 2). Based on contact mechanics and Hertzian contact model, a stiff material (silica) will deform less when in contact with a flat surface. Accordingly, this means that by using silica nanoparticles of the same diameter as polystyrene, smaller sticky patches with higher periodicity are achieved. Further, depositing a mask for NSL made with silica nanoparticles is not trivial as the quality depends on the ability of the particles to not aggregate. Other than polystyrene particles, there are no reports that show the use of different particles to tailor the periodicity and the spot size to fabricate high-throughput arrays.

By using SIMPLE, top-down approaches such as electron-beam (EBL), ion-milling (FIB), and photolithography to fabricate biocompatible arrays could be bypassed. The process allows placing large-scale colloidal crystals for fabricating high-throughput arrays over a variety of substrates that can be processed as per applications. By using solvent: solvent interactions, the interfacial energy of the air/solid/liquid interface is modulated to form monolayers of nanoparticles. This means that by just solubilizing particles in an organic solvent and dropping them on a water droplet, colloidal masks can be easily made and transferred to a substrate. Furthermore, compared to techniques like drop casting and spin coating, SIMPLE can self-assemble colloidal crystals over a large-scale without the need of specialized instrumentation.

The process allows high-throughput single molecule measurements while maintaining deterministic positioning of molecules using nanosphere lithography (NSL) at significantly lower effort. Importantly, arrays made by using SIMPLE are compatible with digital detection techniques, which offer single-copy sensitivity, further enhance the precision and sensitivity of biomolecule detection. With SIMPLE it is now possible to fabricate user defined features to make high-throughput arrays. For instance, with SIMPLE nanoparticles of different size and shape can now be closely packed and transferred on a smooth/rough surfaces of glass/silicon wafer which was relatively difficult to achieve without specialized equipment. The resulting array of patches can be spaced out at a desired distance while preserving the localization of single molecules.

Biomolecule such as DNA origami or proteins can be placed at several microns from each other avoiding their overlap which makes them compatible with standard optical imaging methods and effectively improves the sensitivity and specificity of single molecule measurements. DNA origami nanoarray can be used as a low-cost digital diagnostic technology for many diseases without complex microfluidic devices, bulky microscopes, or PCR machines. By ensuring that each site on the nanoarray contains only a single target molecule, the signal-to-noise ratio of the assay can be significantly improved (as shown in Shetty et al., ACS Nano, 2021, 15 (7), 11441). This increased sensitivity allows for the detection of diseases at earlier stages, when biomarker concentrations are typically lower. Such a platform can rival the sensitivity of droplet digital PCR (1 molecule in 20 μL of sample). Further, DNA origami/protein arrays made with this invention can be used to develop diagnostic tools capable of multiplexed detection of biomarkers or genetic variations while maintaining specificity and can reach extremely small detection levels (sub-femtomolar) two-three orders of magnitude more sensitive than traditional antibody assays.

As shown in the examples, SIMPLE fabricates high-quality, centimeter-scale 2D colloidal crystals with nano-to-micro feature sizes spanning 2 orders of magnitude and on typical substrates like glass. Using a self-assembled monolayer of 1 μm silica nanoparticles as masks for NSL, periodic sticky patches of 100 nm diameter at 1 μm spacing could be obtained. The resulting array facilitates the programmable placement of functionalized DNA origami on addressable substrates. The diameter of the periodic sticky patches may be controlled by the diameter of the nanoparticles forming the colloidal mask. As such, patches of different diameters can be created quickly simply by changing the diameter of the nanospheres used in SIMPLE. The spacing of the sticky patches is controlled by transport kinetics. In some implementations, wide spacing is preferrable to maximize the efficiency of background-mediated 2D transport. DNA origami structures bind weakly to the smooth, passive background and then diffuse across the surface until they “find” a sticky patch. This 2D search is far faster than relying on slow 3D diffusion from the bulk solution, leading to rapid and uniform array saturation.

Equation 1 depicts the equation which can be used to select nanoparticle size for creating the desired size of the periodic sticky patches. The principle of Equation 1 ensures high single-site yield and minimal non-specific adsorption, which are critical metrics for fabrication and quality control. Equation 1 defines the indentation dept of an indenter, where nanosphere diameter is dₙₛ, E_{g} and Eₙₛ are the elastic moduli and v_{g} and vₙₛ are the Poisson's ratios associated with glass and nanospheres, respectively. P is the applied intermolecular pressure.

$$a = \frac{3 P}{16 π} ( \frac{1 - {v}_{g}^{2}}{{E}_{g}} + \frac{1 - {v}_{ns}^{2}}{{E}_{ns}} ) {d}_{ns} \qquad Equation 1$$

The relationship between the nanoparticle's physical size dₙₛ and the chemically defined binding site diameter a is strictly governed by a linear function rooted in contact mechanics: a∝dₙₛ. This physical relationship dictates that the optimal patch diameter must be directly proportional to the physical footprint of nanosphere.

Compared to previous methods, SIMPLE is a low-cost fabrication technique that can be used to produce arrays with diverse applications and can host light radiative and scattering processes associated with nanophotonic systems. Due to its high versatility, good quality, and simplicity, this approach can expand the possibilities of colloidal self-assembly and enhance the performance of applications using colloidal crystals.

The disclosed method has the potential to revolutionize biophysical assays and diagnostics by providing a cost-effective, high-throughput platform for deterministic DNA nanoarrays. By improving the maximum loading efficiency of single DNA origami onto the nanoarray, this technology can facilitate advanced applications in fields such as biosensing, drug discovery, and molecular electronics. Overall SIMPLE represents a significant advancement in biomolecule detection by combining the benefits of NSL with innovative approaches to overcome its limitations, providing a robust, scalable, and cost-effective solution for high-quality single-molecule studies.

In some embodiments, the method of preparing a surface for printing an array comprises combining nanoparticles having Young's modulus of at least 5 GPa with an organic solvent and sonicating the combination of the nanoparticles and the organic solvent to produce colloidal crystals. The method next comprises dropping the colloidal crystals on a glass surface that is submerged in water and drying the colloidal crystals droplets on the glass surface to produce a nanoparticle film over the glass surface. In some aspects, the method prepares a surface in preparation for lithography in which the glass surface is the array surface with the nanoparticle film masking the array surface. In some implementations, the method further comprises covering the nanoparticle film with a silane, which enables production of sticky patches on the glass surface. In some aspects, the nanoparticles for making the colloidal crystals have Young's modulus of about 10 GPa, such as silica nanoparticles. In other aspects, the nanoparticles for making the colloidal crystals have Young's modulus of about 50-200 GPa and are oxides, such as aluminum oxide, zirconium oxide, or titanium oxide.

In some aspects, the silane is an organosilane, such as hexamethyldisilazane (HMDS). In particular embodiments, the silane is a perfluorinated compound, such as trimethoxy (1H,1H,2H,2H)-heptadecafluorodecyl) silane (TMFDS). For the methods of making customized arrays described herein, perfluorinated silanes are preferred over HMDS, a conventional silane for creating sticky patches, because they create a superhydrophobic or oleophobic surface due to its long fluorinated chains and reduces non-specific adsorption of biomolecules and fluorophores significantly. In some aspects, using TMFDS rather than HDMS fabricates a superior surface to enhance single molecule imaging by using a DNA nanoarray.

While silanes are commonly used for surface passivation in single-molecule studies to create a hydrophobic, inert surface that minimizes nonspecific interactions between the biomolecules and the substrate (such as by creating a smooth, non-reactive layer on glass), using silanes for background passivation on a patterned surface is not trivial and involve harsh steps. These harsh steps still leave residues on the array surface that affect DNA origami placement. Additionally, the pattern inversion at nanoscale is challenging to perform without damaging the surroundings to create a small hydrophilic patch surrounded by a large hydrophobic background While the adsorption-reducing properties of perfluorinated compounds have recently been used to create surfaces compatible with single molecule measurements, using perfluorinated compounds to modify patterned surfaces is not a simple switch of molecules. With SIMPLE, silanes can be used to produce sticky spots on a patterned surface to enhance sensitivity of single molecule measurements. Simply covering the nanoparticle film with a silane produces sticky patches is a low stress approach that ensures high fidelity, chemical uniformity and appreciable contrast between hydrophilic sticky patch and inert background making the use of functional silanes practicable for array fabrication.

In some embodiments, the method of preparing a glass surface for printing an array comprises combining nanoparticles having Young's modulus of at least 5 GPa with an organic solvent and sonicating the combination of nanoparticles and the organic solvent to produce colloidal crystals. The colloidal crystals are then dropped on a glass surface that is submerged in water after which the colloidal crystals droplets on the glass surface are dried to produce a nanoparticle film over the glass surface. The method next comprises covering the glass surface having the nanoparticle film with a silane to produce a silane-modified glass surface. The silane is an organosilane, such as a perfluorinated silane. In some aspects, the silane is HMDS or TMFDS. In some implementations, the method of preparing a glass surface for printing an array further comprises removing the nanoparticle film from the glass surface to result in a glass surface comprises sticky patches. In some implementations, the nanoparticle film is removed by sonicating silane-modified glass surface. In some aspects, the method further comprises heating the silane-modified glass surface after removing the nanoparticle film. In some aspects, the nanoparticles for making the colloidal crystals have Young's modulus of about 10 GPa, such as silica nanoparticles. In other aspects, the nanoparticles for making the colloidal crystals have Young's modulus of about 50-200 GPa and are oxides, such as aluminum oxide, zirconium oxide, or titanium oxide.

In some aspects, the method of printing a customizable DNA array comprises combining nanoparticles having Young's modulus of at least 5 GPa with an organic solvent and sonicating the combination of nanoparticles and the organic solvent to produce colloidal crystals. The colloidal crystals are then dropped on a glass surface that is submerged in water after which the colloidal crystals droplets on the glass surface are dried to produce a nanoparticle film over the glass surface. The resulting glass surface is then covered with a silane to produce a modified glass surface and then subjecting the modified glass surface to sonication to remove the nanoparticle film, which results a glass surface with sticky patches, which serve as biomolecule binding sites. The method of printing a customizable DNA array next comprises incubating DNA origami with the glass surface including binding sites thereby hydrolyzing the silane and the DNA origami.

In some implementations of the method of printing a customizable DNA array, the nanoparticles for making the colloidal crystals have Young's modulus of about 50-200 GPa and are oxides, such as aluminum oxide, zirconium oxide, or titanium oxide.

In certain implementations, the nanoparticles have Young's modulus of about 10 GPa. For example, the nanoparticles are silica nanoparticles. In some aspects, the silica nanoparticles have diameters of 1 um. In such implementations, the customizable DNA array is spaced out by 1 um.

In particular implementations of the methods disclosed herein, the method further comprises confirming presence of a layer of nanospheres on the glass surface via diffraction analysis thereby indicating the presence of the nanoparticle film over the glass surface.

### Examples

The present disclosure is further illustrated by the following examples that should not be construed as limiting.

### Example 1: Fabrication of Triangular DNA Origami

Staple strands were purchased from Integrated DNA Technologies, 640 nM each in water and the scaffold strand (single-stranded M13mp18, 400 nM from Bayou Labs, and used without further purification. Scaffold and staple strands were mixed together in 1:5 ratio to target concentrations in 40 mM Tris, 20 mM Acetate, and 1 mM Ethylenediaminetetraacetic acid (EDTA) with a typical pH around 8.6, and 12.5 mM magnesium chloride (MgCl₂) (1×TAE/Mg²⁺). The staple mix (100 μL) were heated to 90° C. for 5 min and annealed from 90° C. to 25° C. at 1° C./min in a PCR machine. Annealed origami were purified using 100 kD molecular weight cut-off filters (MWCO) spin filters (Amicon Ultra-0.5 Centrifugal Filter Units with Ultracel-100 membranes, Millipore, UFC510024) to remove excess staples as they will inhibit DNA origami placement.

Using the protocol below, recovery is generally 40-50%, and staples are no longer visible by agarose gel electrophoresis:
1. Wet the membrane of the spin filter by adding 500 μL 1×TAE/Mg²⁺.
2. Centrifuge at 6000 rcf for 5 min at room temperature (RT), until the volume in the filter is ˜80 μL.
3. Discard the filtrate.
4. Add 100 μL of unpurified origami and 300 μL 1×TAE/Mg²⁺. Spin at 6000 rcf for 5 min at RT.
5. Discard the filtrate.
6. Add 420 μL 1×TAE/Mg²⁺ and spin at 6000 rcf for 5 min at RT.
7. Repeat step (4) two more times.
8. Invert the filter into a clean tube and spin at 6000 ref for 5 min at RT to collect purified origami (˜80 μL).

### Example 2: Fabrication of Array with Silica Nanoparticles

Materials and equipment required:
1. 10×10 mm² coverslips (Ted Pella, 260375-15).
2. Plasma cleaner (Harrick Basic Plasma Cleaner PDC-32G/PDC-3°2G-2)
3. Oven, Hotplate and stirrer (Denville)
4. Desiccator (Hach, Product no. 223830)
5. Branson ultrasonic bath, AFM (Bruker FastScan).
6. Appropriately sized Silica microspheres (NanoXact, 1 um, Nanocomposix)
7. Passivation agent: HMDS (440191-100 mL, Sigma).
8. Plastic Petri dish 35 mm (Corning)
9. N,N-Dimethylformamide anhydrous (DMF), 99.8% (CAS: 68-12-2, Sigma)
10. Dichloromethane (DCM) (Sigma)
11. MilliQ water

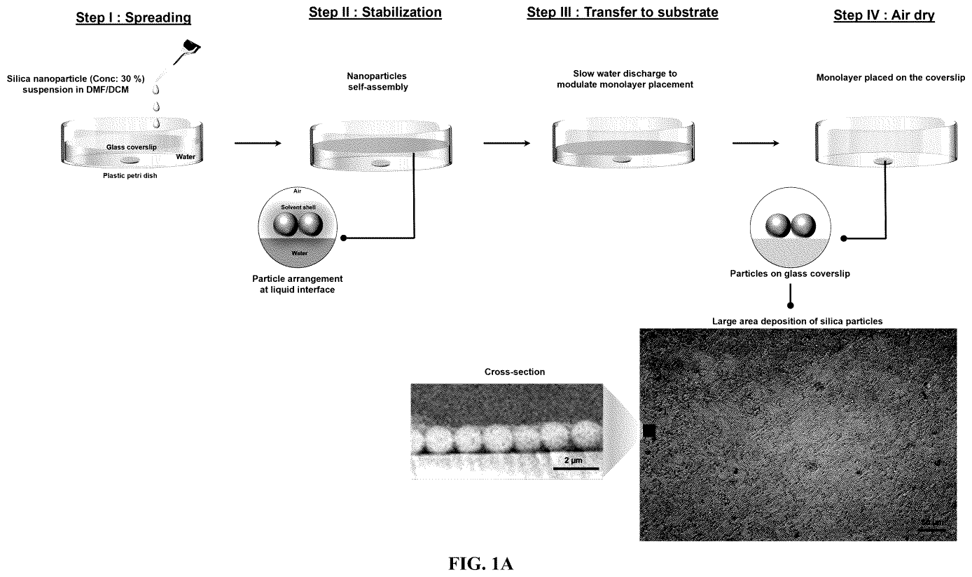

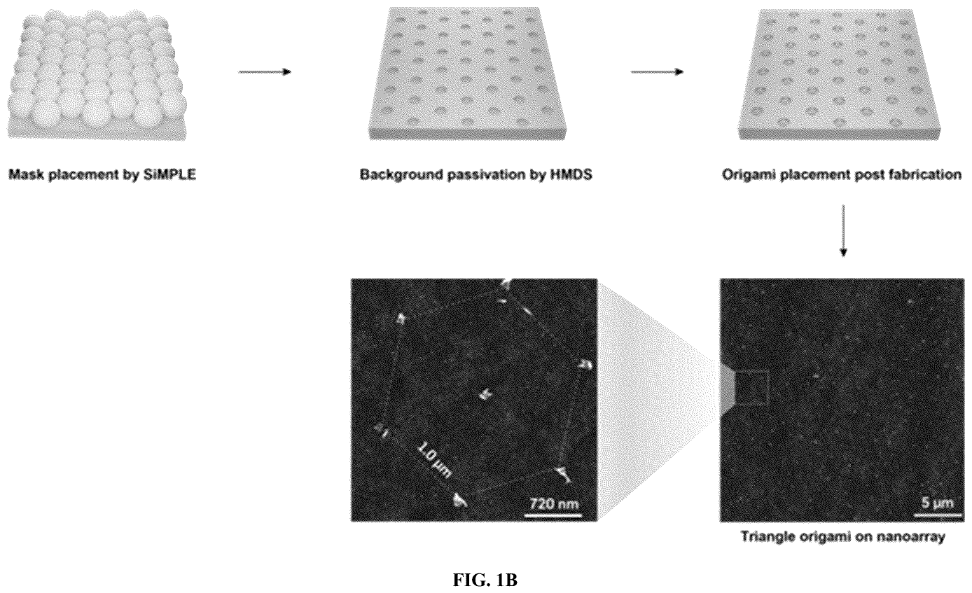

Protocol for binding site creation (FIG. 1A):
1. Wash coverslips with isopropanol (IPA) for 2 min and blow-dry with nitrogen.
2. 10-min air plasma cleaning in Harrick plasma cleaner at ˜18 W (“High” setting) is carried out on the glass.
3. In an Eppendorf tube, weigh 35 mg of 1 μm silica nanoparticles and add 60 μL of DMF. Sonicate for 10 minutes. Pipette 14 μL of Silica: DMF stock and add 6 μL of DCM and sonicate for 10 minutes.
4. In a plastic petri dish (35 mm diameter) place the activated glass coverslip and add 1 ml of MilliQ water on it.
5. Drop 1-2 μL of silica suspension in DMF:DCM mixture on the glass coverslip dipped in MilliQ water. A silica film will form on the air/water interface as the particles pack closely. Once dried, a diffraction pattern can be observed, confirming the existence of a close-packed monolayer/multilayer of nanospheres.
6. Discharge the water from the dish with a pipette, taking care not to introduce vibrations. This carries the self-assembled monolayer slowly on the glass coverslip.
7. Transfer the coated coverslip to a desiccator and place in oven at 150° C. for 15 min to remove excessive moisture.
8. Carry out a 5-min “descum” plasma in air at ˜18 W in Harrick plasma cleaner on the monolayer.
9. Transfer the coverslip to a desiccator, add 8-10 drops of HMDS (in a glass cuvette), and deposit under a vacuum seal for 1 hr.
13. Lift-off silica nanospheres with water sonication in a Branson ultrasonic bath for 30-60 sec and blow dry with a nitrogen gun to create origami binding sites.
15. Bake at 120° C. for 5 min to stabilize the HMDS on the surface.

Protocol for origami placement and washing steps (FIG. 1B).
1. Incubate chips with ˜100-200 pM origami (nominal concentration for 1 μm pitch, concentration inversely proportional to nanosphere size) in ˜40 mM Mg, Tris-HCl (40 mM Tris) buffer (pH—8.3) for 60 min.
2. Wash in ˜40 mM Mg, Tris-HCl (40 mM Tris) buffer (pH—8.3) for 5 min either manually or automatically using a peristaltic pump or shaker in a petri dish.
3. Transfer to ˜40 mM Mg, Tris-HCl (40 mM Tris) buffer (pH—8.3)+0.07% Tween 20 and wash for 5 min.
4. Transfer to ˜35 mM Mg, 10 mM Tris (pH—8.9) to hydrolyze HMDS and lift off origami non-specifically bound to the background, and wash for 5 min.
5. For AFM characterization, transfer to ethanol drying series: 10 seconds in 50% ethanol, 20 seconds in 75% ethanol, and 2 min in 85% ethanol. For optical measurements, skip step 5 but ensure that the patterned coverslip does not dry out by maintaining ˜100 μL of buffer I on the surface.
6. Air-dry, followed by AFM/fluorescence verification of patterning.

DNA origami is placed in the patches created by using a monolayer of 1 μm silica nanoparticles with a periodicity of 1 μm causing a lower PSF overlap and enhanced single-molecule tracking (FIG. 1B). Silica nanoparticles are suspended in organic solvents to avoid aggregation, and an apparatus that allows self-assembly of at air/water interface robustly. Silica particles experience compression at the air/water interface due to changes in interfacial energy at the air/solid/liquid interface and self-assemble in a colloidal crystal. Additionally, the shell of hydrophobic solvent around the silica particles in the crystal promotes close packing allowing interfacial assembly. Then, water discharge allows transfer of the assembled monolayer from the interface onto the substrate without the risk of damage to the colloidal crystal. Array fabrication is initiated by exposing the silanol rich glass substrate with an organosilane that allows passivating the area not covered by the colloidal crystal. Subsequently, activated glass patches are formed after silica nanoparticles are removed. Further, DNA origami is then stabilized onto these patches by a salt bridge and completes the formation of single molecule DNA origami array.

### Example 3: Characterization of DNA Origami Microarray

The DNA origami nanoarray was characterized with atomic force microscopy (AFM) and points accumulation for imaging in nanoscale topography (PAINT) to demonstrate high-throughput and deterministic single molecule experiments.

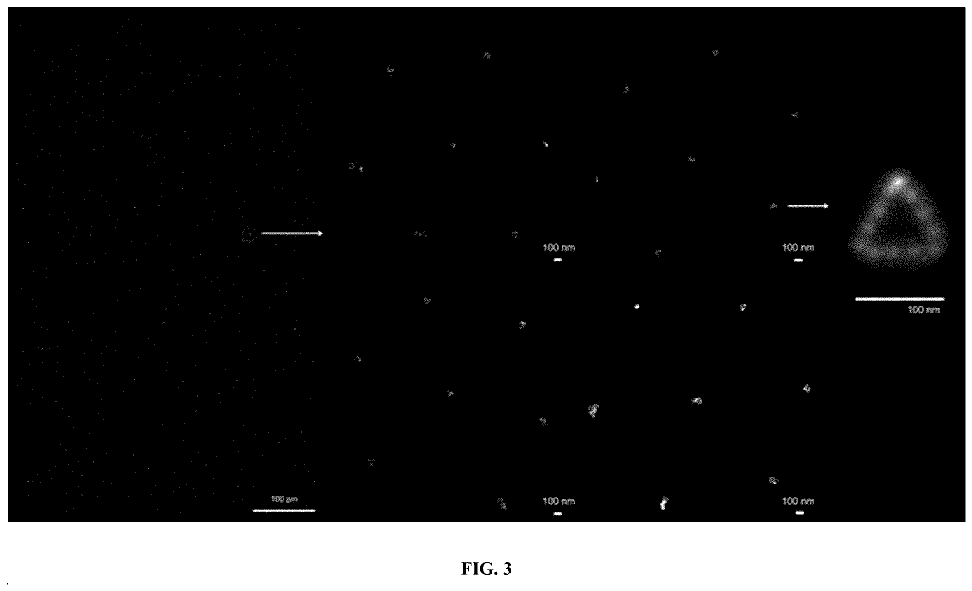

Protocol for super-resolution DNA-PAINT (FIG. 3).
1. Experiments with patterned chips were conducted by sticking the 10 mm×10 mm coverslip onto a double-sided sticky Kapton tape by creating a “flow chamber”.
2. 20 μl of 0.07% Tween-20 in 40 mM Mg²⁺ placement buffer was flown through and incubated for 2 mins.
3. A 1 nM R1-imager solution in placement—Tween buffer, and an oxygen scavenging system (2×, 3×, 5× concentrations of PCA, PCD, and Trolox-Quinone, respectively) was flown through.
3. The flow chamber was sealed with Epoxy resin to prevent drying and avoiding influx of excess oxygen in the chamber.

I/We claim:

1. A method of preparing for a surface for printing an array comprising:
combining silica nanoparticles with an organic solvent;
sonicating the combination of silica nanoparticles and the organic solvent to produce colloidal silica crystals;
dropping the colloidal silica crystals on a glass surface that is submerged in water; and
drying the colloidal silica crystals droplets on the glass surface thereby producing a silica film over the glass surface, wherein the glass surface is an array surface and the silica film masks the array surface in preparation for lithography.

2. The method of claim 1, further comprising covering the silica film with a silane.

3. The method of claim 2, wherein covering the glass surface having the silica film with a silane to produce a silane-modified glass surface.

4. The method of claim 3, wherein the silane is hexamethyldisilazane (HMDS), the method further comprises sonicating HMDS-modified glass surface thereby removing the silica film from the glass surface.

5. The method of claim 4, further comprising heating the HMDS-modified glass surface after removing the silica film.

6. The method of claim 3, wherein the silane-modified glass surface comprises sticky patches spaced out by 1 μm and each sticky patch has a diameter of 100 nm.

7. The method of claim 2, wherein the silane is a perfluorinated compound.

8. The method of claim 1, further comprising confirming presence of layer of silica nanospheres on the glass surface via diffraction analysis.

9. The method of claim 1, wherein the silica nanoparticles have diameters of 1 μm.

10. The method of claim 1, wherein the organic solvent is N,N-dimethylformamide anhydrous (DMF) and/or dichloromethane (DCM).

11. A method of preparing for a surface for printing an array comprising:
combining nanoparticles having Young's modulus of at least 5 GPa with an organic solvent;
sonicating the combination of the nanoparticles and the organic solvent to produce colloidal crystals;
dropping the colloidal crystals on a glass surface that is submerged in water; and
drying the colloidal crystals droplets on the glass surface thereby producing a nanoparticle film over the glass surface, wherein the glass surface is an array surface and the nanoparticle film masks the array surface in preparation for lithography.

12. The method of claim 11, further comprising covering the nanoparticle film with a silane.

13. The method of claim 12, wherein the silane is hexamethyldisilazane (HMDS), a HMDS-modified glass surface is produced.

14. The method of claim 13, further comprising sonicating HMDS-modified glass surface thereby removing the silica film from the glass surface.

15. The method of claim 14, further comprising heating the HMDS-modified glass surface after removing the silica film.

16. The method of claim 11, further comprising confirming presence of a layer of nanospheres on the glass surface via diffraction analysis thereby indicating the presence of the nanoparticle film over the glass surface.

17. The method of claim 11, wherein the nanoparticles have Young's modulus of about 10 GPa or 50-200 GPa.

18. A method of printing a customizable DNA array comprising:
combining nanoparticles having Young's modulus of at least 5 GPa with an organic solvent;
sonicating the combination of nanoparticles and the organic solvent to produce colloidal crystals;
dropping the colloidal crystals on a glass surface that is submerged in water;
drying the colloidal crystals droplets on the glass surface thereby producing a nanoparticle film over the glass surface;
covering the glass surface having the nanoparticle film with a silane to produce a modified glass surface;
removing the nanoparticle film from the modified glass surface to produce a glass surface comprising binding sites; and
incubating DNA origami with the glass surface comprising binding sites thereby hydrolyzing the silane and the DNA origami.

19. The method of claim 18, wherein the nanoparticles are silica nanoparticles having a diameter of 1 μm.

20. The method of claim 19, wherein the customizable DNA array is spaced out by 1 μm.
